(19) United States
(12) Patent Application Publication
(10) Pub. No.: US 2006/0205804 A1
(43) Pub. Date: Sep. 14, 2006

(54) Process for preparing 5-(4-fluorophenyl)-1-[2r,4r)-4-hydroxy-6-oxo-tetrahydro-pyran-2-yl) ethyl]-2-isopropyl-4-phenyl-1h-pyrrole-3-carboxylic acid phenylamide

(76) Inventors: James Douglas Nelson, Mystic, CT (US); Michael Gerard Pamment, Ann Arbor, MI (US)

Correspondence Address: WARNER-LAMBERT COMPANY, 2800 PLYMOUTH RD, ANN ARBOR, MI, 48105, US

(21) Appl. No.: 10/552,747

(22) PCT Filed: Mar. 31, 2004
(86) PCT No.: PCT/IB04/01120

§ 371 Date: Oct. 11, 2005

(30) Foreign Application Priority Data
Apr. 14, 2003 (US) 60462613

(51) Int. Cl. A61K 31/4025; C07D 405/02

(52) U.S. Cl. 514/422; 548/517

(57) ABSTRACT

A method for preparing 5-(4-fluorophenyl)-1[2-((2R,4R)-4-hydroxy-6-oxo-tetrahydro-pyran-2-yl)-ethyl]-2-isopropyl-4-phenyl-1H-pyrrole-3-carboxylic acid phenylamide (I), a key intermediate in the synthesis of atorvastatin calcium, is described.

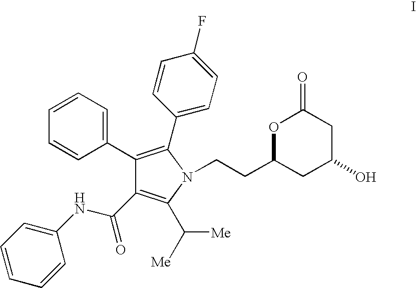

This application claims benefits of U.S. Provisional Application No. 60/462,613, filed on Apr. 14, 2003.

### FIELD OF THE INVENTION

A method for preparing 5-(4-fluorophenyl)-1-[2-((2R,4R)-4-hydroxy-6-oxo-tetrahydro-pyran-2-yl)-ethyl]-2-isopropyl-4-phenyl-1H-pyrrole-3-carboxylic acid phenylamide, a key intermediate in the synthesis of atorvastatin calcium, is described.

### BACKGROUND OF THE INVENTION

5-(4-Fluorophenyl)-1-[2-((2R,4R)-4hydroxy-6-oxo-tetrahydro-pyran-2-yl)-ethyl]-2-isopropyl-4-phenyl-1H-pyrrole-3-carboxylic acid phenylamide (I) is a key intermediate in the synthesis of atorvastatin calcium (Lipitor®), known also by the chemical name [R-(R*,R*)]-2-(4-fluorophenyl)-β,δ-dihydroxy-5-(1-methylethyl)-3-phenyl-4-[(phenylamino)carbonyl]-1H-pyrrole-1-heptanoic acid calcium salt (2:1) trihydrate. Atorvastatin calcium inhibits 3-hydroxy-3-methylglutaryl-coenzyme A reductase (HMG-CoA reductase) and thus is useful as a hypolipidemic and/or hypocholesterolemic agent.

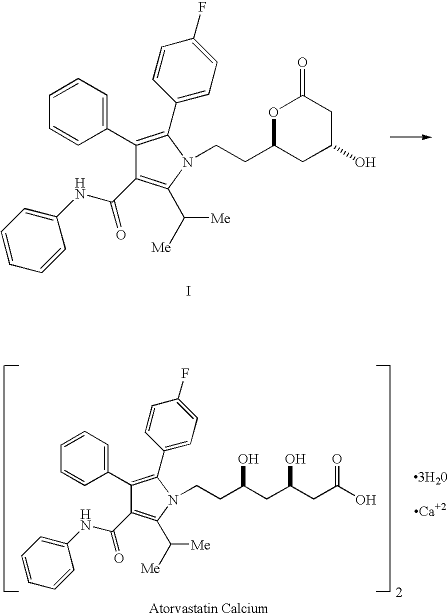

A number of patents have issued disclosing approaches to the preparation of atorvastatin calcium, as well as various analogues, via intermediates such as compound (I). These patents include: U.S. Pat. Nos. 4,681,893; 5,273,995; 5,003,080; 5,097,045; 5,103,024; 5,124,482; 5,149,837; 5,155,251; 5,216,174; 5,245,047; 5,248,793; 5,280,126; 5,397,792; 5,342,952; 5,298,627; 5,446,054; 5,470,981; 5,489,690; 5,489,691; 5,510,488; 5,998,633; and 6,087,511; 5,969,156; 6,121,461; 5,273,995; 6,476,235; U.S. Application Ser. No. 60/401,707 (filed Aug. 6, 2002).

Existing approaches to the preparation of key intermediate (I) presented some shortcomings. For example, one approach relied on the use of a costly chiral raw material ((R)-4-cyano-3-hydroxy-butyric acid ethyl ester), and a low temperature diastereoselective borane reduction.

Scheme 1 summarizes an alternative approach disclosed in U.S. Pat. No. 6,476,235. Hydrogenation of β,δ diketoester 2 in the presence of a chiral ruthenium catalyst under acidic conditions proceeded to give diol 3 in low yields and 1:1 syn:anti diastereoselectivity with respect to the C-3 and C-5 chiral centers.

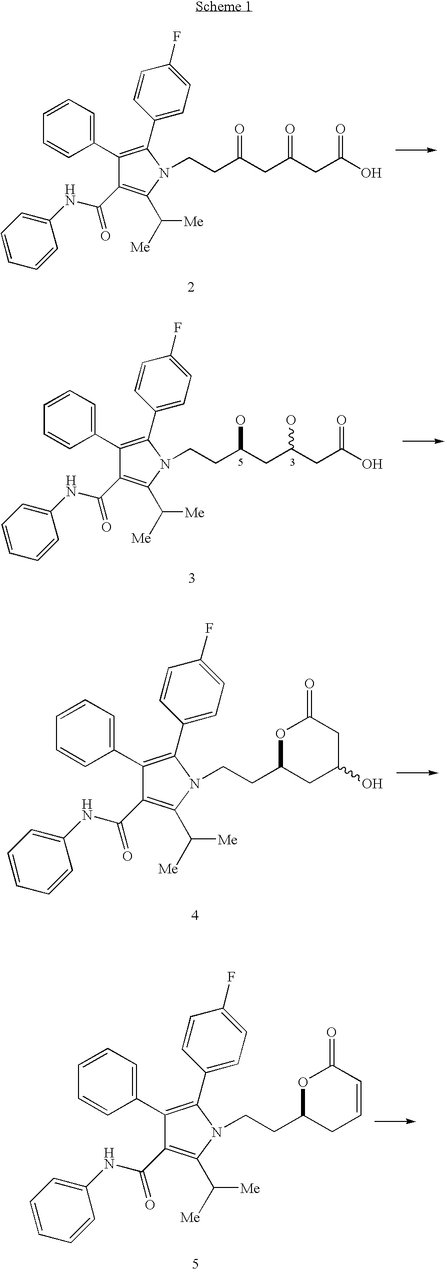

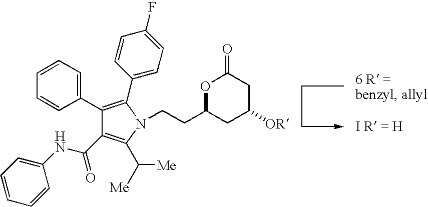

As a preliminary matter, asymmetric hydrogenations of ketones as described above for the transformation of 2 to 3 are known. However, the complexity of the reaction increases in the case of 1,3,5-tricarbonyl systems such as 2, and poor yields and poor stereoselectivities often result. In fact, investigations by Saburi (Tetrahedron, 1997, 1993; 49) and Carpentier (Eur. J. Org. Chem. 1999; 3421) have independently demonstrated low to moderate diastereo- and/or enantio-selectivities for diketoester asymmetric hydrogenations. Furthermore, the fact that the processes disclosed in the literature require high pressure hydrogenation and extended reaction times makes the procedures generally impractical and not amenable to large-scale manufacturing processes where safety, efficiency, and cost are critical considerations.

Referring again to Scheme 1, a number of additional transformations are necessary to reset the stereochemistry of the C-3 center in diol 3 to provide key intermediate (I). These steps include: (a) intramolecular cyclization of 3 to provide lactone 4; (b) elimination of water from lactone 4 using acid to provide α,β unsaturated lactone 5; (c) facial selective Michael addition of allyl or benzyl alcohol to α,β unsaturated lactone 5 to provide saturated lactone 6; and removal of the allyl or benzyl moiety in lactone 6 via hydrogenolysis to provide key intermediate (I).

As a result, a need remains for an approach to the preparation of key intermediate (1) that is efficient, inexpensive, proceeds in a minimum of transformations, and occurs in good yield and high levels of diastereoselectivity.

### SUMMARY OF THE INVENTION

These and other needs are met by the present invention which is directed to a process for the preparation of a compound of formula (I) 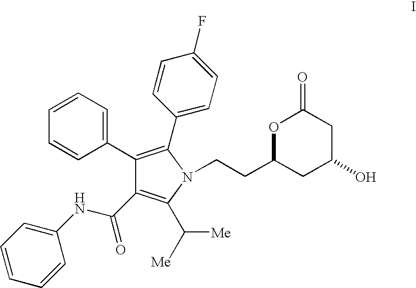
comprising:
(a) contacting in a solvent optionally in the presence of a Lewis acid a compound of formula (II) with 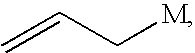
wherein M is SiCl₃, SiMe₃, B(OH)₂, CuLi, MgBr, ZnBr, InBr, SnR₃ wherein R₃ is (C₁-C₆)alkyl, to give a compound of formula (III): 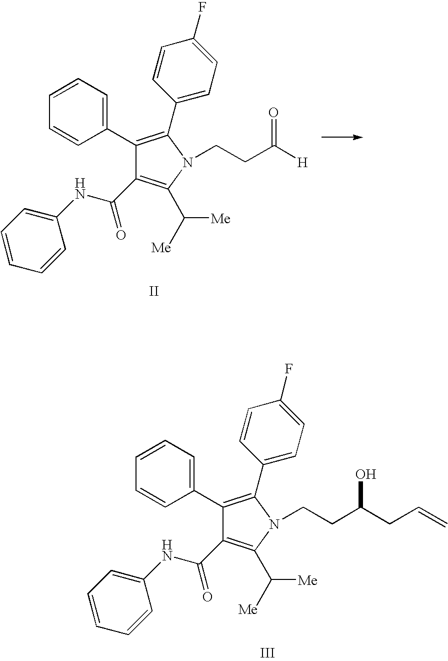
(b) conversion of the compound of formula (III) to an acryloyl ester of formula (IV) in the presence of base using 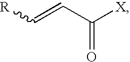
wherein X is Cl, Br, I, or 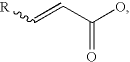
and R is H, (C₁-C₆)alkyl, or phenyl, or an acryloyl activated ester equivalent; 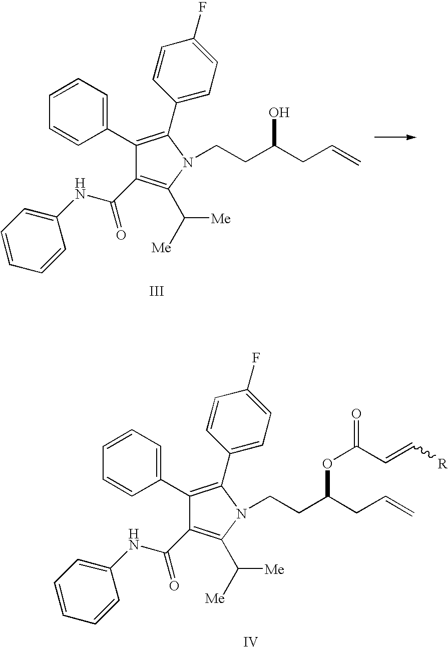
(c) contacting in a solvent the acryloyl ester (IV) with a catalyst to afford 5,6 dihydro pyran-2-one V; 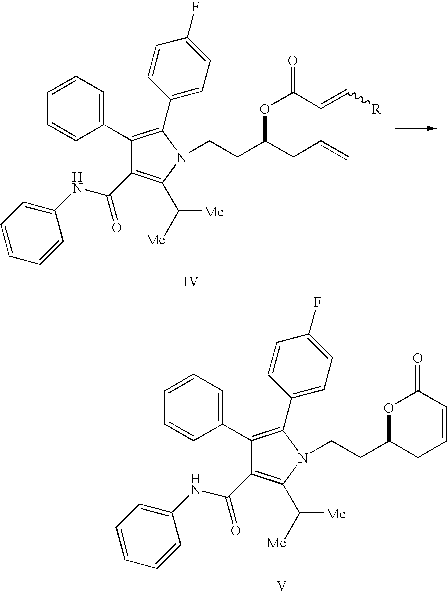
(d) converting the compound of formula (V) to a compound of formula (VI) via facial selective 1,4 addition of allyl or benzyl alcohol; 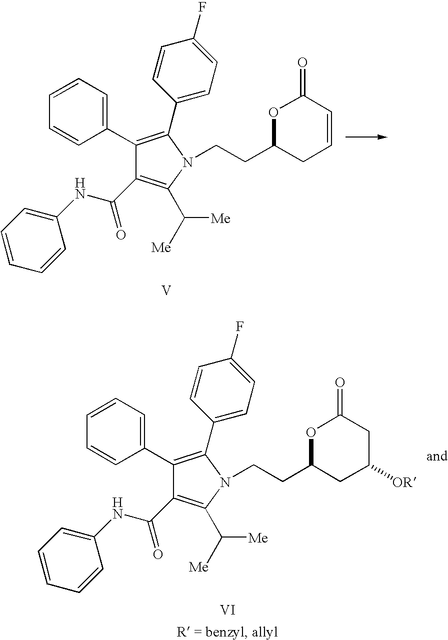
(e) removal of the allyl or benzyl moiety in the compound of formula (VI) via hydrogenolysis to give a compound of formula I.

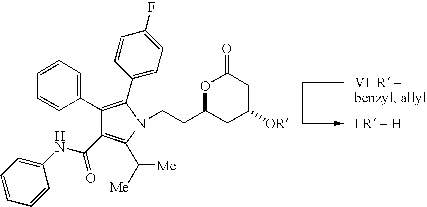

What is also disclosed is a process for the preparation of a compound of formula (I) 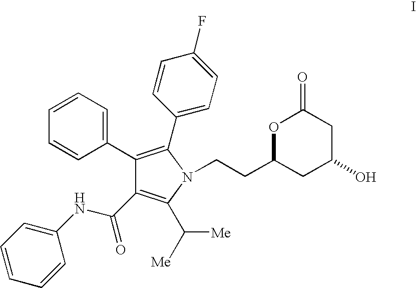
comprising:
(a) contacting in a solvent optionally in the presence of a Lewis acid a compound of formula (II) with 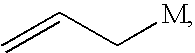
wherein M is SiCl₃, SiMe₃, B(OH)₂, CuLi, MgBr, ZnBr, InBr, SnR₃ wherein R₃ is (C₁-C₆)alkyl, to give a compound of formula (VII): 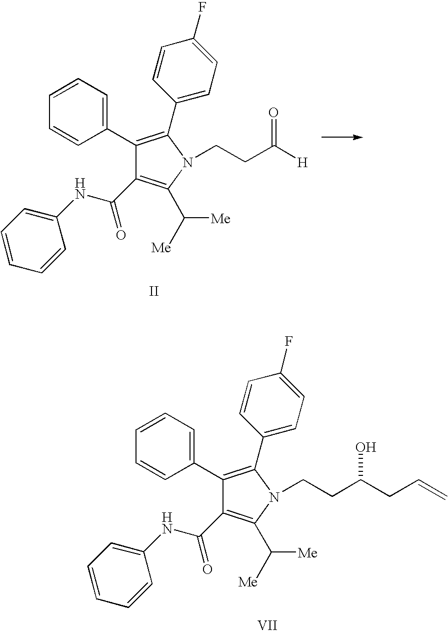
(b) conversion of the compound of formula (VII) with concomitant stereochemical inversion of the homoallylic alcohol center to an acryloyl ester of formula (IV) via Mitsunobu reaction in the presence of acrylic acid or an acrylic acid analogue 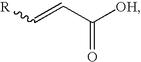
wherein R is H, (C₁-C₆)alkyl, or phenyl, in the presence of base; 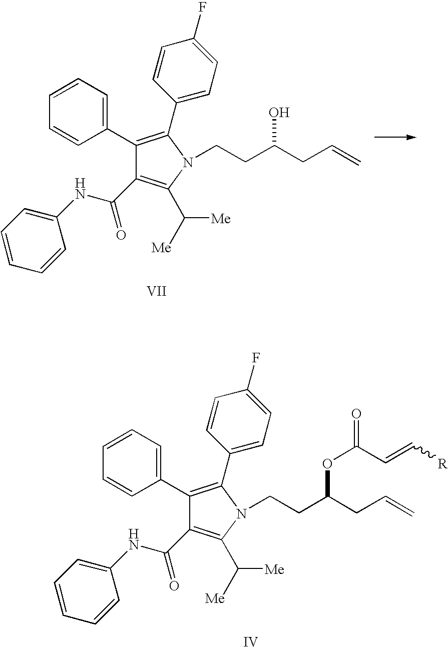
(c) contacting in a solvent the acryloyl ester (IV) with a catalyst to afford 5,6 dihydro pyran-2-one V; 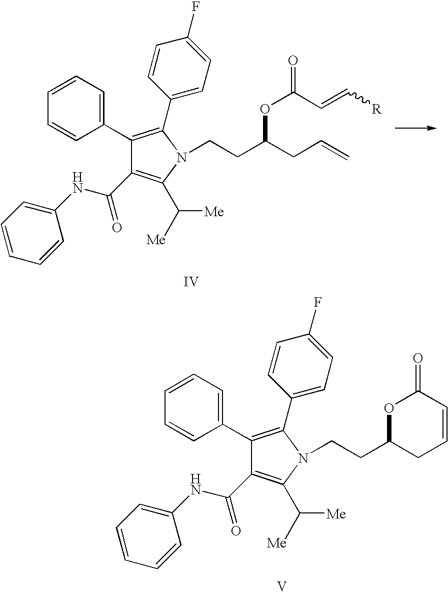
(d) converting the compound of formula (V) to a compound of formula (VI) via facial selective 1,4 addition of allyl or benzyl alcohol; 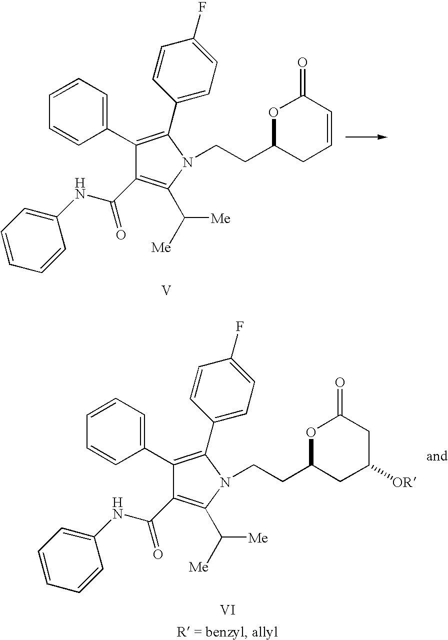
(e) removal of the allyl or benzyl moiety in the compound of formula (VI) via hydrogenolysis to give a compound of formula I.

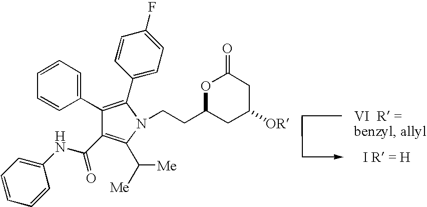

What is further disclosed is a process for the preparation of a compound of formula (I) 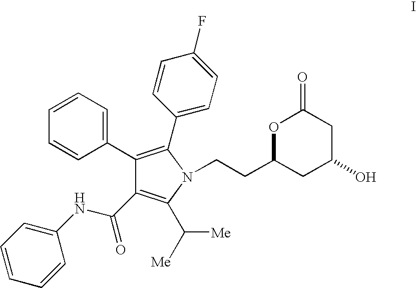
comprising:
(a) contacting in a solvent optionally in the presence of a Lewis acid a compound of formula (II) with 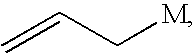
wherein M is SiCl₃, SiMe₃, B(OH)₂, CuLi, MgBr, ZnBr, InBr, SnR₃ wherein R₃ is (C₁-C₆)alkyl, to give a compound of formula (VIII): 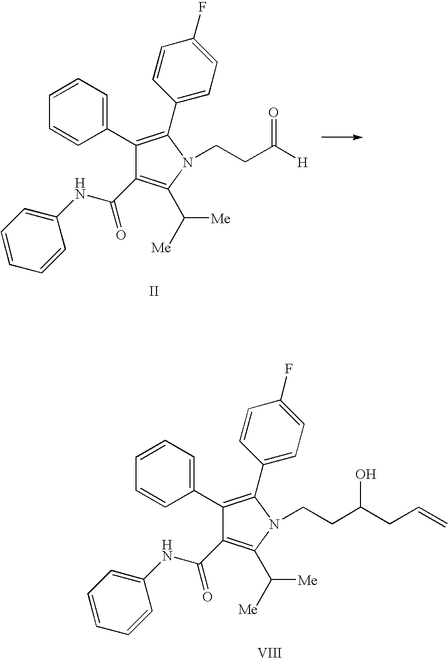
(b) isolating the desired enatiomer (III) from the enantiomeric mixture; 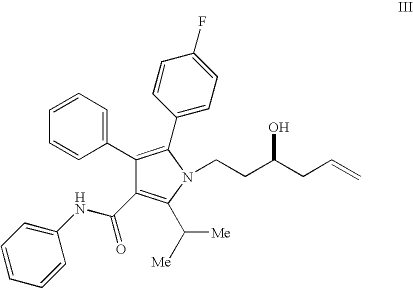
(c) conversion of the compound of formula (III) to an acryloyl ester of formula (IV) in the presence of base using 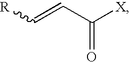
wherein X is Cl, Br, I, or 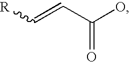
and R is H, (C₁-C₆)alkyl, or phenyl, or an acryloyl activated ester equivalent; 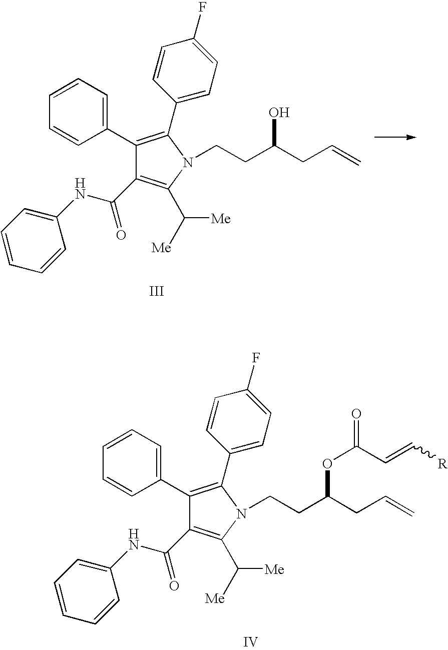
(d) contacting in a solvent the acryloyl ester (IV) with a catalyst to afford 5,6 dihydro pyran-2-one V; 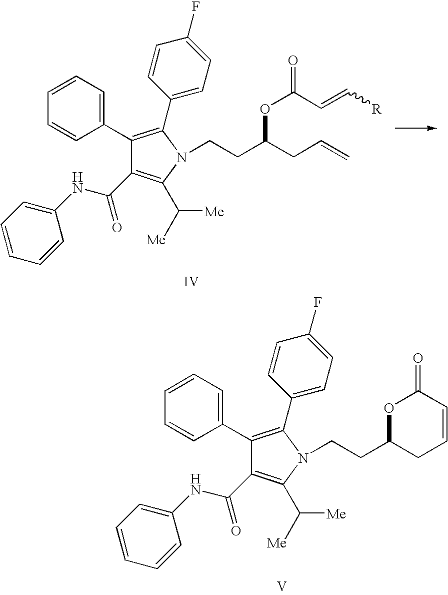
(e) converting the compound of formula (V) to a compound of formula (VI) via facial selective 1,4 addition of allyl or benzyl alcohol; 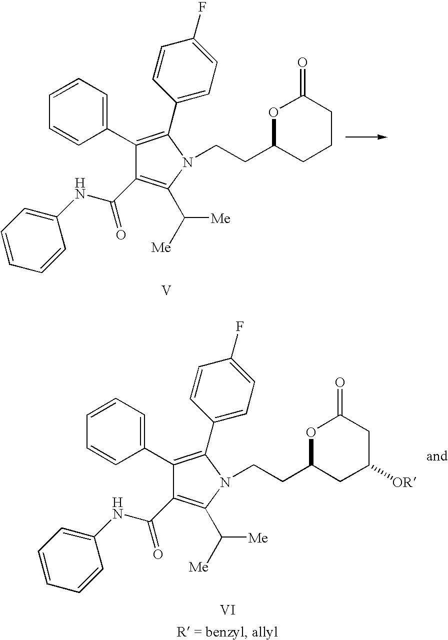
(f) removal of the allyl or benzyl moiety in the compound of formula (VI) via hydrogenolysis to give a compound of formula I.

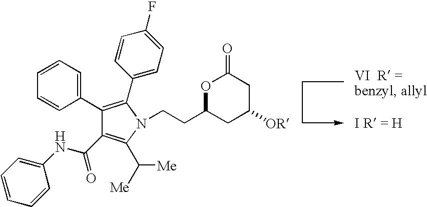

What is also provided is a process for the preparation of a compound of formula III 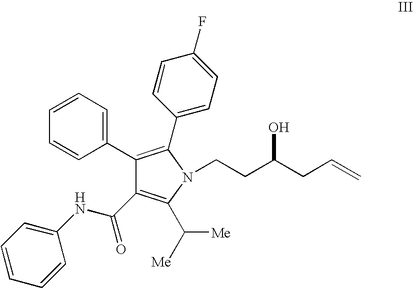
comprising:
(a) contacting a compound of formula (II) with an allenylboronic ester to give a compound of formula (XI): 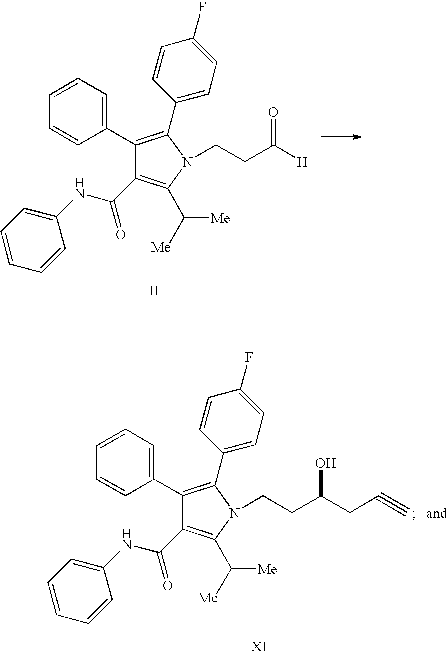
(b) hydrogenation of the compound of formula (XI) to provide a compound of formula III

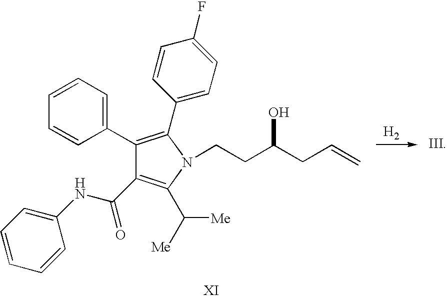

What is also provided is a process for the preparation of a compound of formula VII 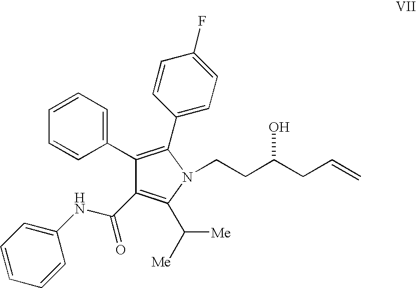
comprising:
(a) contacting contacting (II) with an allenylboronic ester to give a compound of formula (XII): 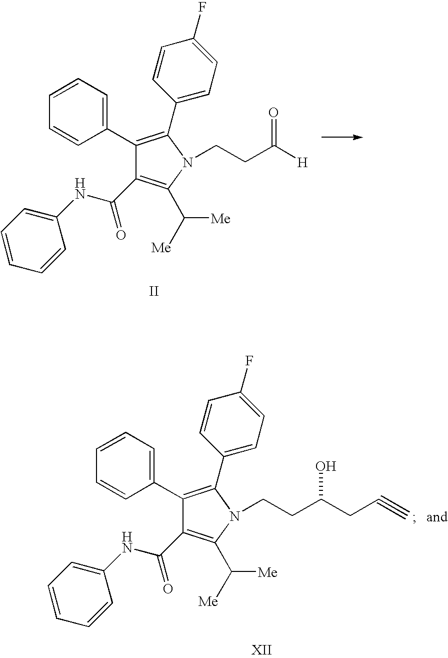
(b) hydrogenation of the compound of formula (XII) to provide the compound of formula (VII)

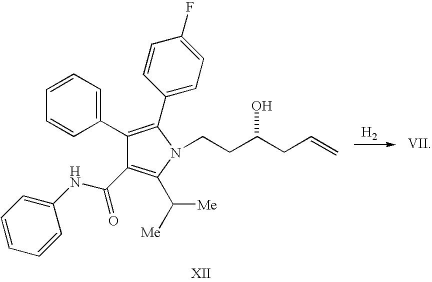

What is also provided is a process for the preparation of a compound of formula VIII 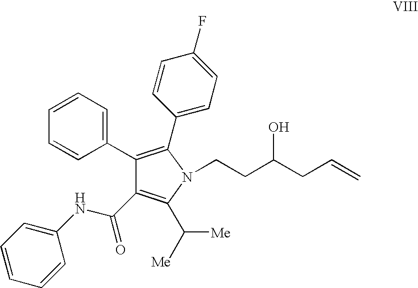
comprising:
(a) contacting a compound of formula (II) with allenylboronic acid or an allenylboronic ester to give a compound of formula (XIII): 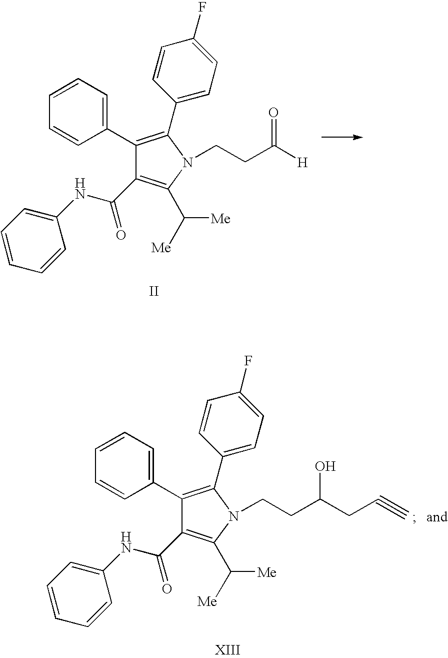
(b) hydrogenation of the compound of formula (X) to provide VII

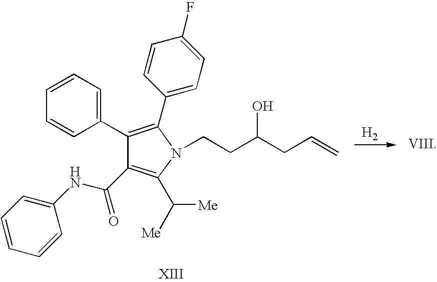

What is also provided is a compound of formula III.

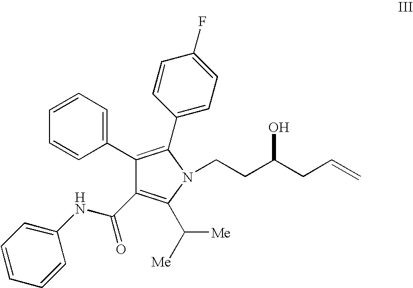

What is also provided is a compound of formula VIII.

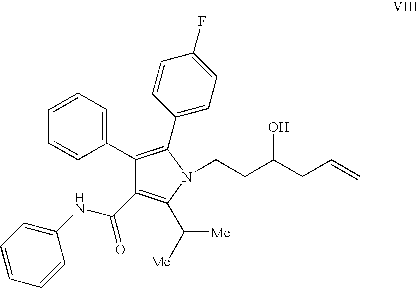

What is also provided is a compound of formula VII.

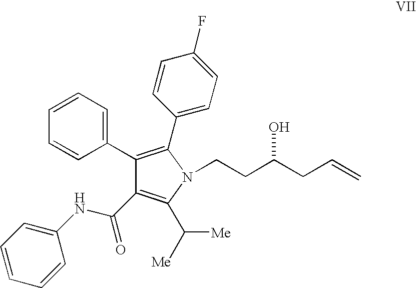

What is also provided is a compound of formula IX.

What is also provided is a compound of formula IV.

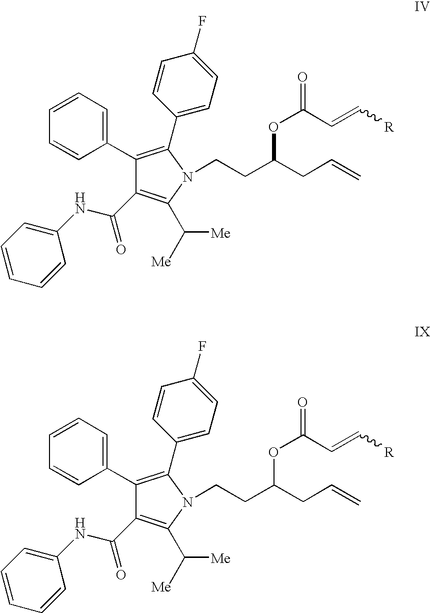

What is also provided is a compound of formula X.

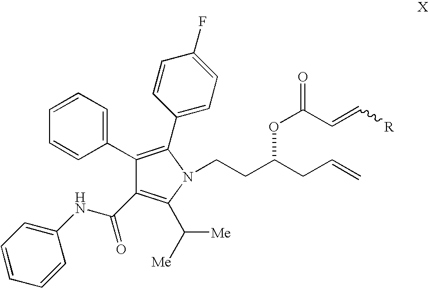

What is also provided is a compound of formula XI.

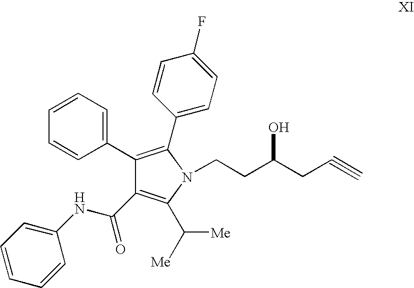

What is also provided is a compound of formula XII.

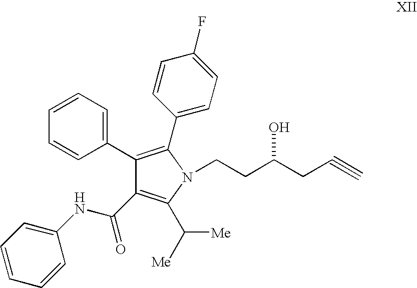

What is also provided is a compound of formula XIII.

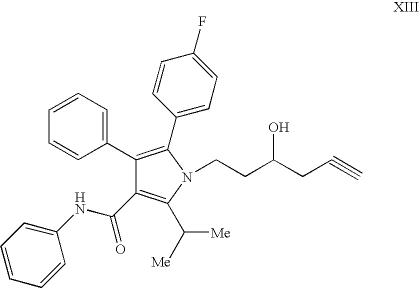

As disclosed herein, we surprisingly and unexpectedly found that 5,6 dihydro pyran-2-one (V) can be obtained conveniently from acryloyl ester (IV) via a mild and efficient one-step ring-closing metathesis reaction in the presence of a homogeneous catalyst. The reaction proceeds in good yields at temperatures below approximately 60° C. and atmospheric pressure. The invention process is thus safer and more efficient in large scale than earlier approaches, because it avoids the need for specialized high-pressure equipment. In addition, a minimum number of transformations are necessary to incorporate the C-3 hydroxy group, and the overall number of steps needed to convert the compound of formula (II) to key intermediate (I) is minimized. Moreover, the invention process avoids the use of a costly, chiral raw material ((R)-4-cyano-3-hydroxy-butyric acid ethyl ester), and a low temperature diastereoselective borane reduction, as was necessary in earlier approaches to the preparation of key intermediate (I).

### DETAILED DESCRIPTION OF THE INVENTION

### DEFINITIONS

(C₁-C₆)alkyl means both straight and branched groups; but reference to an individual radical such as “propyl” embraces only the straight chain radical, a branched chain isomer such as “isopropyl” being specifically referred to. Thus, (C₁-C₆)alkyl can be methyl, ethyl, propyl, isopropyl, butyl, iso-butyl, sec-butyl, pentyl, 3-pentyl, or hexyl.

The term “approximate” as used herein in reference to a quality, condition, or amount, means the value specified in relation to the quality, condition, or amount is nearly exact, or nearly or more or less as specified.

If ranges defined by two endpoints are provided for a particular value disclosed herein (relating, for instance, to reaction temperature, time, concentration, or stoichiometry), that range is intended to cover the endpoints and all real values, both fractions and integers between the endpoints.

Invention Process

As a preliminary matter, the compounds prepared by the invention process disclosed herein may have one or more chiral centers and may exist in and be used or isolated in optically active and racemic forms. Thus, it is to be understood that the processes of the present invention can give rise to any racemic or optically-active forms, or mixtures thereof, as described herein. It is to be further understood the products of the invention process can be isolated as racemic, enantiomeric, or diastereomeric forms, or mixtures thereof. Purification and characterization procedures for such products are known to those of ordinary skill in the art, and include recrystallization techniques, as well as chiral chromatographic separation procedures as well as other methods.

The invention process disclosed herein is summarized in Scheme 2. Although it depicts the synthesis of the desired chiral series, the sequence of reactions disclosed in Scheme 2 can be modified as needed (i.e., by use of chiral versus non-chiral auxiliaries, Lewis acids, or ligands, depending on the reaction type) to provide both chiral and non-chiral products.

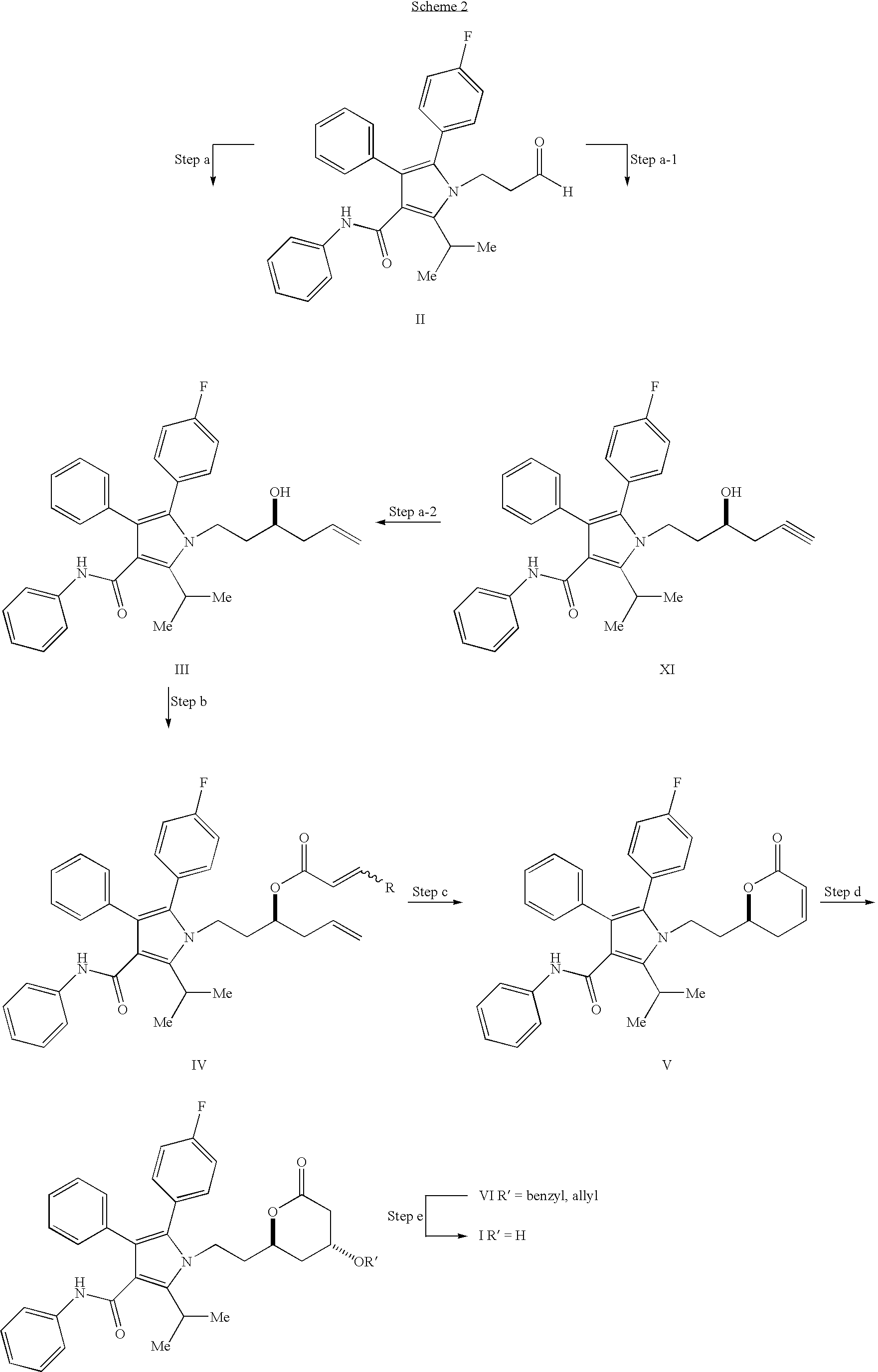

The invention process commences with step (a)or step (a-1)/(a-2). In step (a), allylation of aldehyde (II) provides homoallylic alcohol III. In step (a-1)l(a-2), addition of an allenylboronic ester to aldehyde (II) provides homopropargylic alcohol XI. Hydrogenation of homopropargylic alcohol (XI) in step (a-2) provides homoallylic alcohol III.

In step (b), the hydroxyl group in compound (III) is allowed to react with acryloyl chloride to provide acryloyl ester IV. In step (c), a ring-closing metathesis reaction provides key intermediate V. In step (d), the C-3 hydroxyl group, protected as the corresponding benzyl or allylic ether, is appended stereoselectively to the compound V. Removal of the protecting group and hydrogenolysis provides compound I.

The synthetic sequence disclosed in Scheme 2 is described in greater detail in the following sections.

Step (a)

In step (a) of the invention process, the aldehyde (II) undergoes allylation using 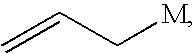
wherein M is SiCl₃, SiMe₃, B(OH)₂, CuLi, MgBr, ZnBr, InBr, SnR₃ wherein R₃ is (C₁-C₆)alkyl,to provide homoallylic alcohol III. Methods for performing the allylation of aldehydes are well known and are widely available to the skilled artisan and typically rely on the use of a Grignard reagent (e.g., allyl magnesium bromide) or a Grignard reagent equivalent, such as an allyl zinc, allyl borane (such as allyl dihydroxyborane), an allylboronic ester, allyl cuprate, allyl tin (such as allyl tri-n-butylstannane), allyl silane (such as allyl trichlorosilane or allyl triemthylsilane), or allyl indium reagent. Methods for preparing and using these reagents are well known to the skilled artisan based on reports in the chemical literature. Many are also commercially available.

A Lewis acid optionally may be used to mediate asymmetric induction and/or mediate the allylation reaction. The use of Lewis acids is well known in organic synthesis. See Hisashi Yamamoto, Lewis acids in Organic Synthesis (2002). In a non-chiral embodiment of the invention process, a non-chiral Lewis acid may be used to catalyze the allylation process, as depicted in Scheme 3, to provide homoallylic alcohol (VIII) as a racemic mixture. In this series, the desired enantiomer (III) can be isolated using procedures available to the skilled artisan, for instance, by chromatographic separation using a chiral stationary phase or resolution of the racemic form by established recrystallization techniques.

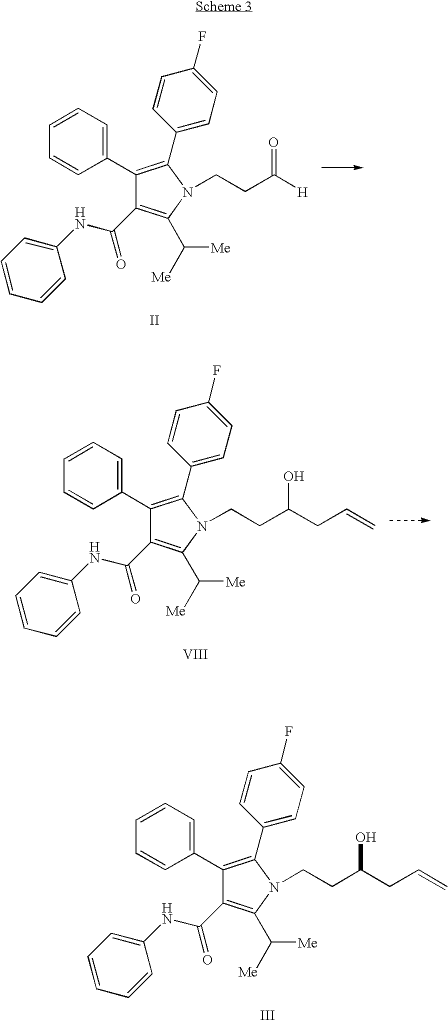

In another embodiment of step (a) of Scheme 2, a chiral Lewis acid can be used to control the enantioselectivity, as well as to mediate the process. In one embodiment of the invention process, a Lewis acid generated in situ, derived from boron tribromide and (S,S)-1,2-diamino-1,2-diphenylethane bis-toluenesulfonamide, was employed to provide a 94.4% enantiomeric excess of the desired S isomer as shown in Scheme 2.

In yet another embodiment of step (a) of Scheme 2, as depicted in Scheme 4, the opposite enantiomer (VII) also can be synthesized by choosing an appropriate chiral Lewis acid. In this reaction variant, compound (VII) is readily converted to the preferred enantiomer (III) under conditions available to the skilled artisan. For instance, Mitsunobu-type reaction of (VII) in the presence of triphenyl phosphine, tributyl phosphine or the like, diethylazodicarboxylate or an equivalent reagent such as di-ispopropylazodicarboxylate or 1,1′(azodicarbonyl)dipiperidine, and a carboxylic acid such as benzoic, formic, or acetic acid, will provide ester IIIa. Ester IIIa readily may be converted to homoallylic alcohol (III) under reduction or hydrolysis conditions available to the skilled artisan. Alternatively, acrylic acid can be used as the acid component of the Mitsunobu-reagent system, to provide homoallylic ester (III) in one pot.

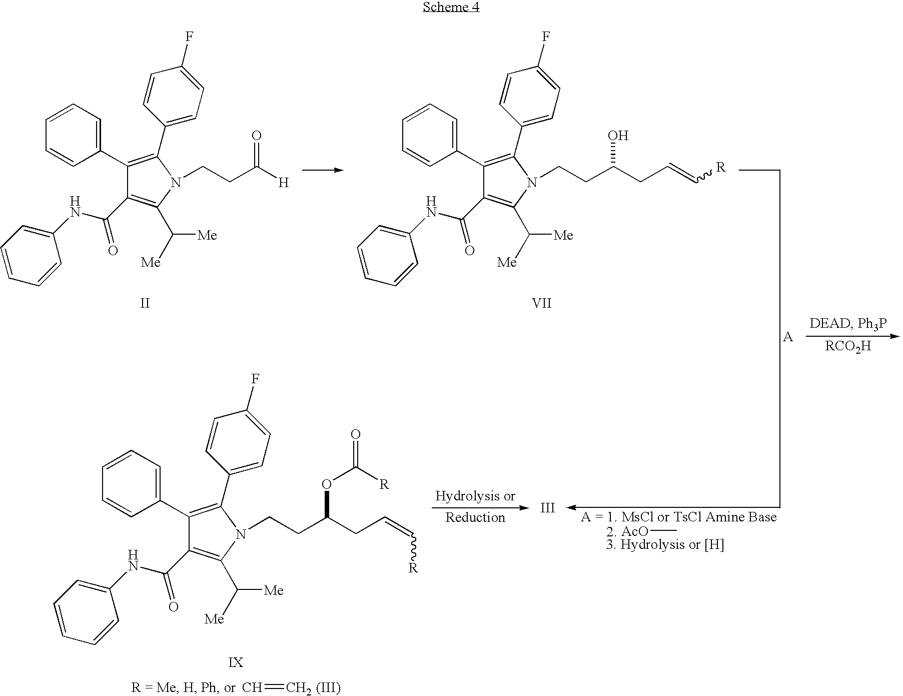

An alternative approach to the conversion of compound (VII) to compound (III) is also depicted in Scheme 4 and requires conversion of the alcohol moiety in compound (VII) to a leaving group such as a mesylate or tosylate, for instance, by mesylation or tosylation or the like, followed by nucelophilic displacement with an appropriate oxygen nucleophile such as acetate to provide the ester. Reduction or hydrolysis of the ester provides compound III. Methods are readily available to the skilled artisan for performing this sequence of transformations.

It is worth noting that a Lewis acid is not a necessary reaction component in some cases, as when allyl trichlorosilane is employed in the presence of an amino alcohol or diamine. See Kinnaird, et. al., J. Am. Chem. Soc. 2002, 124, 7920. It is also worth noting that the reaction proceeds in the presence of a Lewis Base when allyl trichlorosilane is used. See Denmark, et. al., J. Am. Chem. Soc. 2001, 123, 9488.

In one embodiment of step (a) of the invention process, the stoichiometry of the allylation reaction components is typically approximately:

1.0 equivalent of aldehyde;

1.05-1.5 equivalents of Lewis acid; and

1.05-1.5 equivalents of allyl Grignard reagent or allyl Grignard equivalent reagent.

In another embodiment of the invention process, the stochimetry of the allylation reaction is typically approximately:

1.0 equivalent of aldehyde;

1.05-1.3 equivalents of Lewis acid; and

1.05-1.3 equivalents of allyl Grignard reagent or allyl Grignard equivalent reagent.

In still another embodiment of the invention process, the stochimetry of the allylation reaction is typically approximately:

1.0 equivalent of aldehyde;

1.05-1.2 equivalents of Lewis acid; and

1.05-1.2 equivalents of allyl Grignard reagent or allyl Grignard equivalent reagent.

In one embodiment of the invention process, the concentration of the aldehyde in dichloromethane is typically approximately 0.05 to 0.125 mM.

In another embodiment of the invention process, the concentration of the aldehyde in dichloromethane is typically approximately 0.075 to 0.10 mM.

In still another embodiment of the invention process, the concentration of the aldehyde in dichloromethane is typically approximately 0.08 to 0.09 mM.

The temperature of the allylation reaction typically is in the range of approximately −78° C. to approximately room temperature, or 25° C.

The time required for the allylation reaction typically is in the range of approximately 12 to approximately 24 hours, or until the conventional analytical techniques such as TLC or HPLC indicate that the reaction has achieved completion.

In general, the time and temperature parameters of the allylation reaction will vary somewhat depending on reaction concentration and stoichiometry. The skilled artisan can readily adjust the reaction parameters as needed to optimize reaction yields on a run-by-run basis.

In a typical procedure employing a chiral Lewis acid generated in situ, (S,S)-1,2-diamino-1,2-diphenylethane bis-toluenesulfonamide is dissolved in a polar non-protic solvent. Polar non-protic solvents useful in the first step of the invention process include, for example, dichloromethane, chloroform, 1,1,1 trichloroethane, 1,1,2 trichloroethane and the like. Typically, dichloromethane is used. The mixture of the chrial auxiliary in solvent is then cooled to 0° C. and BBr₃ is added dropwise at a rate sufficient to maintain the reaction temeperature at 0° C. The resulting mixture is stirred at 0° C. for 10 minutes and then is allowed to warm to room temperature, is stirred for an additional 40 minutes, and is then concentrated in vacuo. The residue is taken up in a solvent such as dichloromethane and concentrated in vacuo again to remove excess boron tribromide. The residue is then dissolved in dichloromethane and the resulting mixture is cooled to 0° C. To this cooled reaction mixture is added an allyl metal reagent such as tributylstannane, after which the resulting mixture is warmed to ambient temperature and stirred for approximately 1 to approximately 4 hours. The mixture is cooled to −78° C. and the aldehyde (II) dissolved in dichloromethane is added dropwise. The mixture is then stirred for an additional 12 to 24 hours. Conventional workup and purification affords the desired product.

Step (a) Alternative: Steps (a-1)and (a-2)

An alternative to step(a) is depicted in step (a-1) and step (a-2) and involves the addition of an allenylboronic ester to aldehyde (II) to provide the homopropargylic alcohol XI, followed by hydrogenation.

Step (a-1)

The reaction of allenylboronic esters with aldehydes, and more notably, the use of chiral allenylboronic esters in enantoselective synthesis, is well known to the skilled artisan. See R. Haruta, M. Ishiguro, N. Ikeda, and H. Yamamoto. J. Am. Chem. Soc. 1982, 104, 7667; N. Ikeda and H. Yamamoto. J. Am. Chem. Soc. 1986, 108, 483;E. J. Corey, C.-M. Yu, and D.-H. Lee. J. Am. Chem. Soc. 1990, 112, 878.

In a non-chiral context, treatment of aldehyde (II) with allenylboronic acid, prepared as described in N. Ikeda and H. Yamamoto. J. Am. Chem. Soc. 1986, 108, will give rise homopropargylic alcohol XIII, as depicted in Scheme 5.

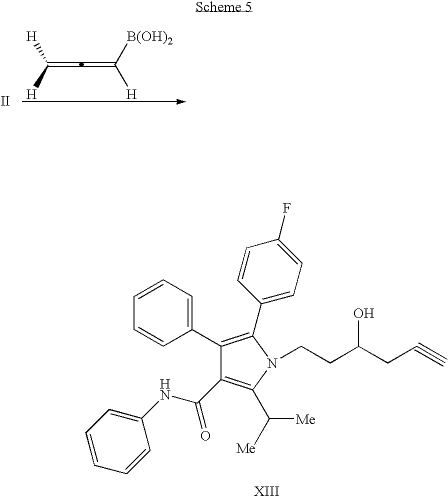

In a chiral context, depending on the chiral auxiliary employed, either homopropargylic acid (XI) or (XII) may be prepared, as shown in Scheme 6. For example, as described in R. Haruta, M. Ishiguro, N. Ikeda, and H. Yamamoto. J. Am. Chem. Soc. 1982, 104, 7667 or N. Ikeda and H. Yamamoto. J. Am. Chem. Soc. 1986, 108, 483, the addition of a chiral allenylboronic ester generated from allenylboronic acid using (+)-dialkyl tartrate, such as diethyl, di-isopropyl, dicyclopentyl, dimenthyl, dicyclododecyl, or di-2,4-dimethyl-3-pentyl tartrate, will give rise to homopropargylic acid XI. The use of a (−)-dialkyl tartrate will provide homopropargylic acid XII. Other variants of the approach are known to the skilled artisan and include, for example, the procedure described in E. J. Corey, C.-M. Yu, and D.-H. Lee. J. Am. Chem. Soc. 1990, 112, 878.

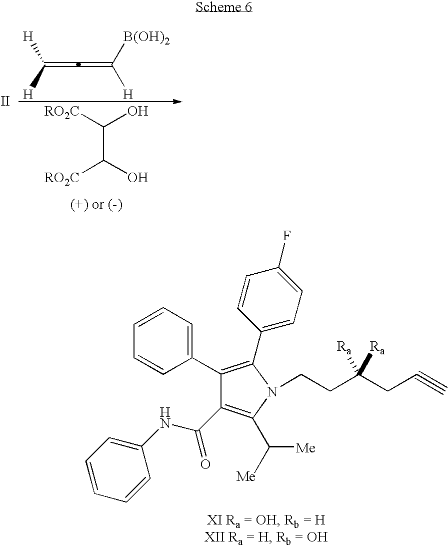

In a typical procedure, allenylboronic acid can be combined with (+)-diethyl tartrate in tetrahydrofuran as described in N. Ikeda and H. Yamamoto. J. Am. Chem. Soc. 1986, 108. The tetrahydrofuran can be removed in vacuo, leaving the allenylboronic ester, which can be used without further purification. Aldehyde (II) can be added to a solution of the allenylboronic ester in toluene or the like at from approximately −80 to approximately −10° C. Conventional work-up (extraction into diethyl ether, drying over magnesium sulfate, and concentration in vacuo) and purification (silica gel column chromatography) will give rise to homopropargylic alcohol XI. The same procedure, except employing (−)-diethyl tartrate, will give rise to homopropargylic alcohol XII.

Step (a-2)

Hydrogenation of homopropargylic alcohol (XI) will provide homoallylic alcohol III. Conditions for effecting the hydrogenation are well known to the skilled artisan and may be carried out under heterogeneous conditions or homogeneous conditions. The heterogeneous catalyst known as Lindlar's catalyst, which is a lead-modified palladium-CaCO₃ catalyst, is generally employed for this transformation (See H. Lindlar and R. Dubuis. Org. Synth. 1973, V, 880).

Step (b)

In step (b) of the invention process, homoallylic alcohol (III) is converted to the acryloyl ester (IV) upon reaction with 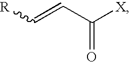
wherein X is Cl, Br, I, or 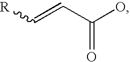
and R is H, (C₁-C₆)alkyl, or phenyl, or upon reaction with an acryloyl activated ester equivalent, in the presence of base. “Acryloyl activated ester equivalent” means an acryloyl mixed anhydride wherein X is a sterically hindered moiety such as 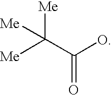
It also means an acryolyl mixed anydride generated from a chloroformate, or from carbonyl di-imidazole. The reaction of an alcohol with an acid chloride, anhydride, or mixed anhydride is well known in the art (See, for example, Junzo Otera, Esterification: Methods, Reactions, and Applications, Wiley-VCH, Weinheim, 2003). In general, the reaction requires the use of an amine base such as triethylamine, di-isopropylethylamine, DBU, or DBN, or the like, in the presence of a catalytic amount of 4-(dimethylamino)pyridine (MAP). The transformation proceeds smoothly without protection of the amide nitrogen. Alternative procedures may also be used, such as relying on the use of carbodiimide coupling reagents.

In one embodiment of the invention process, the stoichiometry of the reaction components in the esterification reaction is typically approximately:

1.0 equivalent of homoallylic alcohol;

1.05-1.5 equivalents of acryolyl chloride;

1.05-1.5 equivalents of amine base; and

0.1 to 0.5 equivalent DMAP.

In another embodiment of the invention process, the stoichiometry of the reaction is typically approximately:

1.0 equivalent of homoallylic alcohol;

1.1-1.4 equivalents of acryolyl chloride;

1.1-1.4 equivalents of amine base; and

0.15 to 0.4 equivalent DMAP.

In still another embodiment of the invention process, the stoichiometry of the reaction is typically approximately:

1.0 equivalent of homoallylic alcohol;

1.15-1.3 equivalents of acryolyl chloride;

1.15-1.3 equivalents of amine base; and

0.2 to 0.3 equivalent DMAP.

In one embodiment of the invention process, the concentration of the acrylate ester in dichloromethane is typically approximately 0.01 to 0.05 mM.

In another embodiment of the invention process, the concentration of the acrylate ester in dichloromethane is typically approximately 0.015 to 0.045 mM.

In still another embodiment of the invention process, the concentration of the aldehyde in dichloromethane is typically approximately 0.02 to 0.04 mM.

The temperature of the esterification reaction typically is in the range of approximately room temperature, or approximately −5° C., to approximately 20° C.

The time required for the reaction typically is in the range of approximately 4 to approximately 24 hours, or until the conventional analytical techniques such as TLC or HPLC indicate that the reaction has achieved completion.

In general, the time and temperature parameters of the reaction will vary somewhat depending on reaction concentration and stoichiometry. The skilled artisan can readily adjust the reaction parameters as needed to optimize reaction yields on a run-by-run basis.

In a typical procedure, 5-(4-Fluoro-phenyl)-1-(3-hydroxy-hex-5-enyl)-2-isopropyl-4-phenyl-1H-pyrrole-3-carboxylic acid phenylamide (III) is dissolved in a polar non protic solvent such as dichloromethane. The reaction is cooled to −5° C. and an amine base such as triethylamine is added, along with a catalytic amount of 4-(dimethyl amino)pyridine (DMAP). To this cooled reaction mixture is slowly added acryloyl chloride dissolved in dichloromethane. Additional triethylamine and/or DMAP may be added as needed to drive the reaction to completion. The reaction mixture is quenched, worked up, and purified under conventional conditions to provide IV.

Step (c)

In step (c) of the invention process, acryloyl ester (IV) undergoes ring-closing metathesis in the presence of a homogeneous organometallic catalyst to provide 5,6 dihydro pyran-2-one IV. A number of metal catalysts are available for the purpose of performing ring-closing metathesis reactions, including, for instance, commercially available bis(tricyclohexylphosphine) benzylidene ruthenium (IV) dichloride A (“Grubbs' Catalyst) in the presence or absence of Ti(O-iPr)₄ (G. C. Fu and R. H. Grubbs, J. Am. Chem. Soc., 1992, 114, 5426; See also A. K. Ghosh and H. Lei, J. Org. Chem., 2000, 65, 4779 and references cited therein; Grubbs, R. H. and Chang, S., Tetrahedron Lett., 1998, 54, 4413; Cossy, J., Pradaux, F. and BouzBouz, S., Org. Lett., 2001, 3, 2233; Held, C., Frohlich, R. and Metz, P., Ang. Chem. Int. Ed. Eng., 2001, 40, 1058; Reddy, M. V., Rearick, J. P., Hoch, N. and Ramachandran, P. V., Org. Lett., 2001, 3, 19; P. V. Ramachandran, M. V. Reddy, and H. C. Brown, J. Indian. Chem. Soc., 1999, 76, 739; Greer, P. B. and Donaldson, W. A., Tetrahedron Lett., 2000, 41, 3801; Ghosh, A. and Wang, Y. Tetrahedron Lett., 2000, 41, 2319; Ghosh, A. and Bilcer, G., Tetrahedron Lett., 2000, 41, 1003; Ramachandran, P. V., Reddy, M. V., and Brown, H. C., Ghosh, A. and Wang, Y. Tetrahedron Lett., 2000, 41, 583; Ghosh, A., and Liu, C., Chem. Commun., 1999, 1743; Ghosh, A. K., Capiello, J., and Shin, D. Ghosh, A. and Wang, Y. Tetrahedron Lett., 1998, 39, 4651; Reddy, M. V., Yucel, A., Ramachandran, P. V., J. Org. Chem., 2001, 66, 2512).

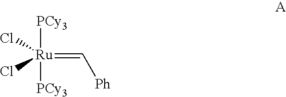

An alternative catalyst for use in the metathesis reaction of the invention process is B.

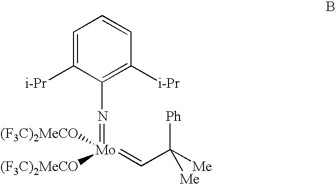

See,.e.g., Schrock, R. R., Murdzek, J. S., Bazan, G. C., Robbins, J., DiMare, M., and O'Regan, M. B., J. Am. Chem. Soc. 1990, 112, 3875; Bazan, C., Khosravi, E., Schrock R. R., Feast, W. J., Gibson, V. C., O'Regan, M. B., Thomas, J. K., Davis, W. M., J. Am. Chem. Soc., 1990, 112, 8378; Bazan, C., Oskam, J. H., Cho, H. N., Park, L. Y., Schrock, R. R., J. Am. Chem. Soc., 1991, 113, 6899.

An additional alternative reaction approach is to generate the catalyst in situ, as provided in Morgan, J. P. and Grubbs, R. H., Org. Lett., 2000, 2, 3153; Huang, J., Stevens, E. D., Nolan, S. P., Petersen, J. L., J. Am. Chem. Soc., 1999, 121, 2674; Furstner, A., Thiel, O., Ackerman, L., Schanz, H.-J. and Nolan, S. P. J. Org. Chem., 2000, 65, 2204). Such catalysts include, for example, 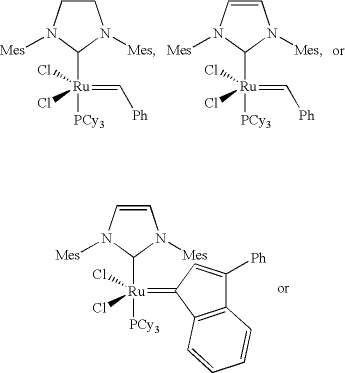
the like.

In one embodiment of the invention process, the stoichiometry of the reaction components is typically approximately:

1.0 equivalent of acrylate ester; and

0.025-0.075 equivalents of catalyst.

In another embodiment of the invention process, the stoichiometry of the reaction is typically approximately:

1.0 equivalent of acrylate ester; and

0.04-0.06 equivalents of catalyst.

In still another embodiment of the invention process, the stoichiometry of the reaction is typically approximately:

1.0 equivalent of acrylate ester; and

0.045-0.055 equivalents of catalyst.

In one embodiment of the invention process, the concentration of the acrylate ester in dichloromethane is typically approximately 0.05 to 0.125 mM.

In another embodiment of the invention process, the concentration of the acrylate ester in dichloromethane is typically approximately 0.08 to 0.11 mM.

In still another embodiment of the invention process, the concentration of the acrylate ester in dichloromethane is typically approximately 0.09 to 0.10 mM.

The temperature of the metathesis reaction typically is in the range of approximately 25° C. to approximately 50° C.

The time required for the reaction typically is in the range of approximately 4 to approximately 24 hours, or until the conventional analytical techniques such as TLC or GC indicate that the reaction has achieved completion.

In general, the time and temperature parameters of the reaction will vary somewhat depending on reaction concentration and stoichiometry. The skilled artisan can readily adjust the reaction parameters as needed to optimize reaction yields on a run-by-run basis.

In a typical procedure, (IV) is dissolved in dichloromethane. The mixture is degassed under vacuum, then purged with nitrogen. The mixture is then warmed to reflux, Grubb's catalyst A 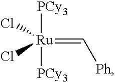
(CAS #1246047-72-3) in degassed dichloromethane is added. The mixture is allowed to stir at reflux for approximately 12 to approximately 24 hours. Workup and purification under conventional procedures provides V.

Benefits of the approach to (V) via this ring closing reaction, particularly when a homogeneous catalyst is employed, include:
Smaller quantities of catalyst are needed because of typically the high turnover numbers of homogeneous catalysts, increasing efficiency and reducing the overall cost of the transformation;
The ability to run production-scale reactions in a minimal amount of solvent, thus reducing waste management requirements and environmental concerns;
The ability to run the reactions at room temperature and atmospheric pressure, thus reducing the need to use specialized pressurized production-scale apparatus, and simplifying work-up procedures; and
An overall reduction in the number of synthetic steps needed to make the compound stereoselectively.
   Step (d)

Step (d) of the invention process is disclosed in U.S. Pat. No. 6,476,235 (corresponding to U.S. Ser. No. 10/015,558, allowed as of Jul. 22, 2002).

Step (e)

Step (e) of the invention process is disclosed in U.S. Pat. No. 6,476,235 (corresponding to U.S. Ser. No. 10/015,558, allowed as of Jul. 22, 2002) provides 1, which is a convenient precursor to atorvastatin.

### EXAMPLES

The following examples are intended to illustrate various embodiments of the invention and are not intended to restrict the scope thereof.

### Example 1

### Preparation of 5-(4-Fluoro-phenyl)-1-(3-hydroxy-hex-5-enyl)-2-isopropyl-4-phenyl-1H-pyrrole-3-carboxylic acid phenylamide (III)

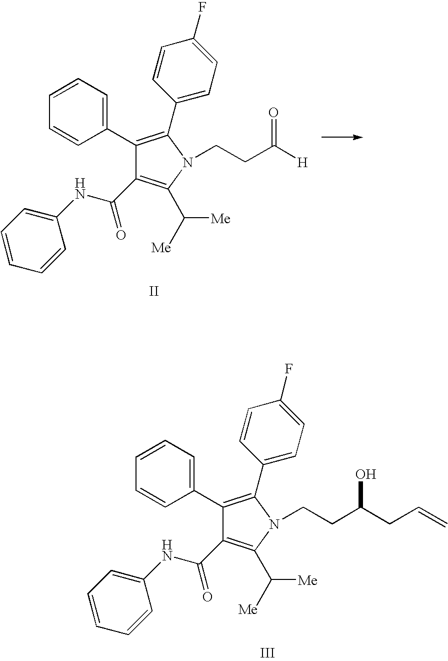

A flask was charged with 1.25 g (2.4 mmol, 1.14 equiv) of (S,S)-1,2-diamino-1,2-diphenylethane bis-toluenesulfonamide, followed by 20 ml of CH₂Cl₂. The resulting mixture was cooled to 0° C. and 2.0 mL (2.33 mmol, 1.1 equiv) of BBr₃ was added dropwise. The reaction was stirred at 0° C. for 10 minutes and then allowed to warm to ambient temperature and stirred for an additional 40 minutes. The reaction mixture was concentrated in vacuo and taken up in 8 ml of CH₂Cl₂ and concentrated in vacuo. Again, 20 ml of CH₂Cl₂ was added to the reaction mixture and the resulting solution was cooled to 0° C. To the cooled reaction mixture was added 0.75 ml (2.31 mmol, 1.1 equiv) of allyl tributylstannane, after which the mixture was warmed to ambient temperature and stirred for two hours. The reaction was cooled to −78° C. and 0.96 g (2.1 mmol, 1.0 equiv) of aldehyde (II) dissolved in 2.5 ml of CH₂Cl₂ was added dropwise. After three hours and an additional 0.5 g of aldehyde dissolved in 2.5 ml of CH₂Cl₂ was added dropwise and stirred overnight. The reaction was quenched by the addition of 10 ml of pH 6.2 phosphate buffer. The organic layer was washed with 10 ml of saturated aqueous sodium chloride and was then condensed. The resulting mixture was dissolved in 10 ml of CH₂Cl₂ and diluted with 40 ml of heptane. The chiral diamino auxiliary was recovered in 97% yield. The filtrate was stirred with 20 ml of 33% aqueous KF to remove tin salts. The organic layer was dried over MgSO₄ and condensed followed by dissolving in 50 ml of EtOAc filtering and again condensing. This was repeated with an additional 12 ml of EtOAc and finally condensing to give 0.98 g (95% yield) of an oil. LC-MS API-ES negative ionization M 496.3 and M−1 495.3; LC-MS API-ES positive ionization M 496.3 and M+1 497.3.

### Example 2

### Preparation of Acrylic acid 1-{2-[2-(4-fluoro-phenyl)-5-isopropyl-3-phenyl-4-phenylcarbamoyl-pyrrol-1-yl]-ethyl}-but-3-enyl ester (IV)

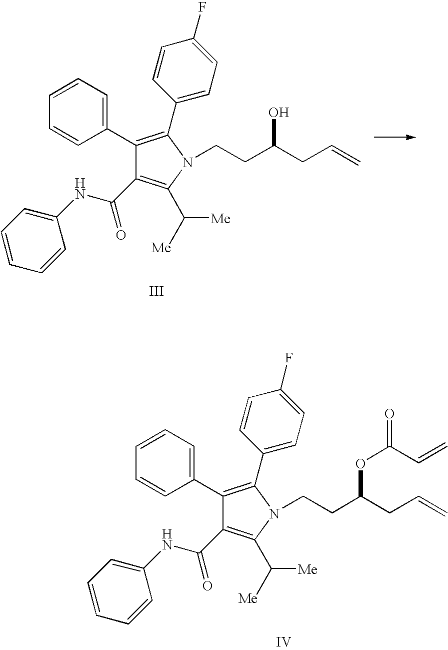

To a flask was added 0.98 g (1.98 mmol, 1 equiv) of ⁵-(4-Fluoro-phenyl)-1-(3-hydroxy-hex-5-enyl)-2-isopropyl-4-phenyl-1H-pyrrole-3-carboxylic acid phenylamide (III) and 10 ml of CH₂Cl₂. The reaction was cooled to −5° C. and 0.33 ml (2.38 mmol, 1.2 equiv) of triethylamine and 0.048 g ((0.396 mmol, 0.2 equiv) of 4-(dimethyl amino)pyridine were added. To the cooled reaction mixture was slowly added 0.19 ml (2.38 mmol, 1.2 equiv) of acryloyl chloride dissolved in 10 ml of CH₂Cl₂. An additional 0.33 mil of triethylamine and 0.048 g of 4-(dimethyl amino)pyridine was added to the reaction mixture, followed by 0.19 ml of acryloyl chloride dissolved in 3 ml of CH₂Cl₂. The reaction was quenched with 20 ml of aqueous NaHCO₃. The organic layer was washed with 20 ml of aqueous NaHCO₃, followed by saturated aqueous NaCl, dried over MgSO₄, and concentrated in vacuo to give 0.9 g (88% yield) (IV) as an orange solid.

HPLC Retention time 17.0 minutes wavelength at 254 nm. Acetonitrile:water w/0.1% formic acid 60:40 (0 to 5 min) 100:0 (15 to 22 min) 60:40 (25 min), YMC ODS-AQ S5; 120 A; 4.6×250 mm; flow rate at 1 ml/min and column temperature at 30° C.

### Example 3

### Preparation of 5-(4-Fluoro-phenyl)-2-isopropyl-1-[2-(6-oxo-3,6-dihydro-2H-pyran-2-yl)-ethyl]-4-phenyl-1H-pyrrole-3-carboxylic acid phenylamide (V)

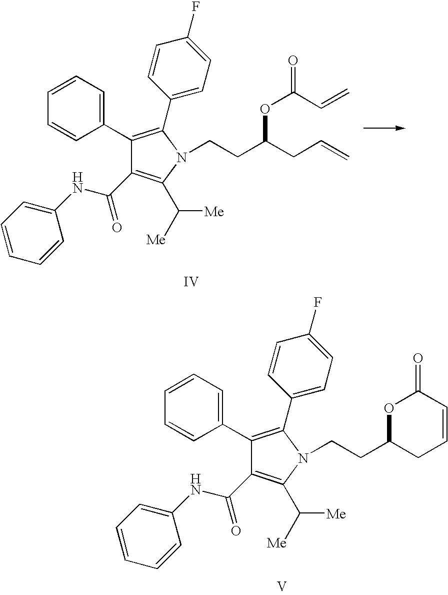

To a flask was added 0.9 g (0.8 mmol, 1 equiv) of acrylate ester in 45 ml of CH₂Cl₂. The mixture was degassed a single time under vacuum followed by nitrogen. The reaction was warmed to reflux. To the reaction mixture was added 0.035 g (0.04.mmol, 0.05 equiv) of Grubb's catalyst (CAS #1246047-72-3) in 5 ml of degassed solvent. The reaction was allowed to stir at reflux for 19 hours. The mixture was condensed and subjected to silica gel flash chromatography eluting with 10% EtOAc/heptane with a gradient increased to 40% EtOAc/heptane. After condensing suitable fraction 0.3 g of a white solid (72% yield) was isolated.

HPLC Retention time 13.3 minutes wavelength at 254 nm. Acetonitrile:water w/0.1% formic acid 60:40 (0 to 5 min) 100:0 (15 to 22 min) 60:40 (25 min), YMC ODS-AQ S5; 120 A; 4.6×250 mm; flow rate at 1 ml/min and column temperature at 30° C.

Chiral HPLC analysis hexane:isopropanol 90:10 Chirapak AD; 4.6×250 mm; flow rate at 1 ml/min and column temperature at 30° C.

(S) retention time 16.6 min

(R) retention time 19.1 min

Ratio of 97.22:2.78

94.4% enantiomeric excess.

All patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

1. A process for preparing a compound of formula (I) 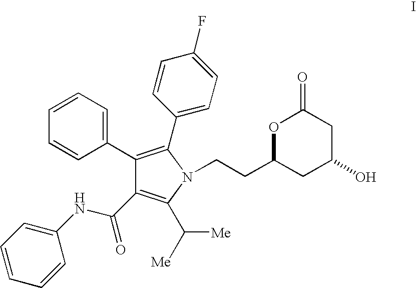
comprising:
(a) contacting in a solvent optionally in the presence of a Lewis acid a compound of formula (II) with 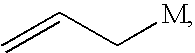
wherein M is SiCl₃, SiMe₃, B(OH)₂, CuLi, MgBr, ZnBr, InBr, SnR₃ wherein R₃ is (C₁-C₆)alkyl, to give a compound of formula (III): 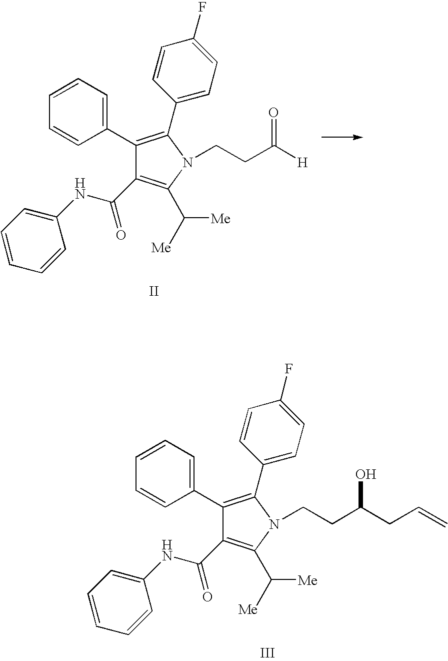
(b) conversion of the compound of formula (III) to an acryloyl ester of formula (IV) in the presence of base using
wherein X is Cl, Br, I, or 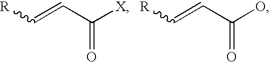
and R is H, (C₁-C₆)alkyl, or phenyl, or an acryloyl activated ester equivalent; 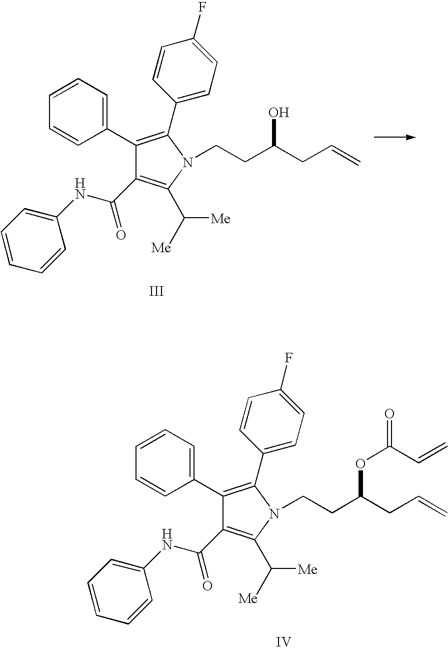
(c) contacting in a solvent the acryloyl ester (IV) with a catalyst to afford 5,6 dihydro pyran-2-one V; 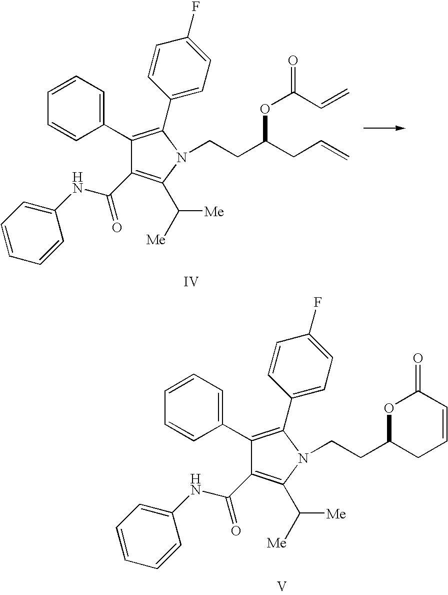
(d) converting the compound of formula (V) to a compound of formula (VI) via facial selective 1,4 addition of allyl or benzyl alcohol; 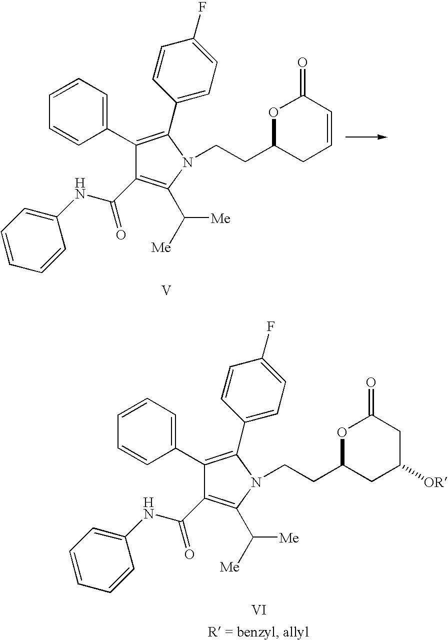
R′=benzyl, allyl and
(e) removal of the allyl or benzyl moiety in the compound of formula (VI) via hydrogenolysis to give a compound of formula I.

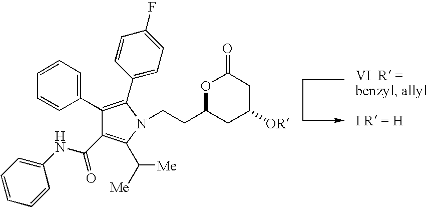

2. The process of step (a) of claim 1, wherein 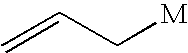
is allyl tri-n-butylstannane, allyl trimethylsilane, allyltrichlorosilane, allyl magnesium bromide, or allyl zinc bromide, optionally used in the presence of an amino alcohol or diamine or a Lewis Base.

3. The process of step (a) of claim 1 carried out in the presence of a nonchiral or chiral Lewis acid, optionally generated in situ from boron tribromide and (S,S)-1,2-diamino-1,2-diphenylethane bis-toluenesulfonamide.

4. The process of step (b) of claim 1 wherein the base is an amine base selected from the group consisting of triethyl amine, N,N dimethyl amino pyridine, DBU, and DBN optionally in the presence of a catalytic amount of DMAP and the polar nonprotic solvent is dichloromethane.

5. The process of step (c) of claim 1, wherein the catalyst is 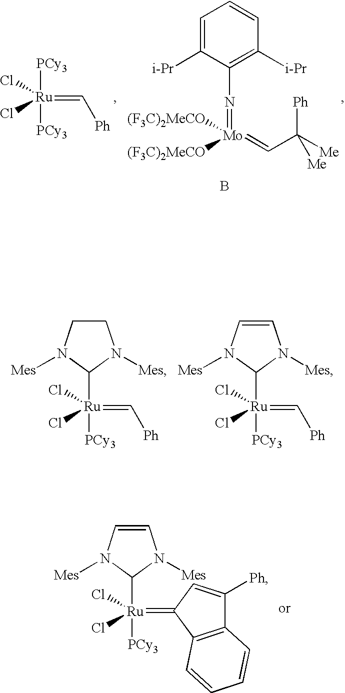
benzylidene[1,3-bis(2,4,6-trimethylphenyl)-2-imidazolidinylidene]dichloro (tricyclohexylphosphine)ruthenium.

6. A process for the preparation of a compound of formula (I) 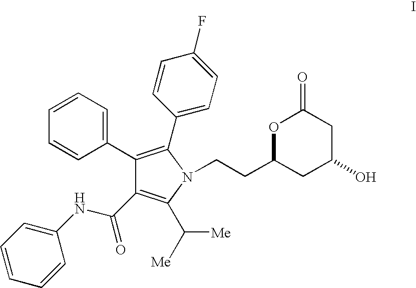
comprising:
(a) contacting in a solvent optionally in the presence of a Lewis acid a compound of formula (II) with 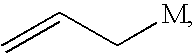
wherein M is SiCl₃, SiMe₃, B(OH)₂, CuLi, MgBr, ZnBr, InBr, SnR₃ wherein R₃ is (C₁-C₆)alkyl, to give a compound of formula (III): 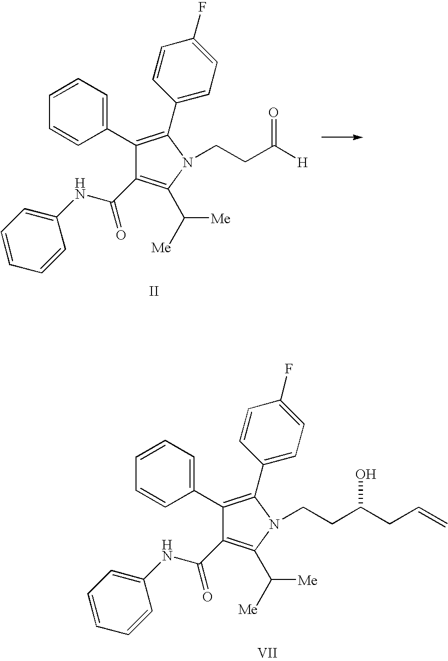
(b) conversion of the compound of formula (VII) with concomitant stereochemical inversion of the homoallylic alcohol center to an acryloyl ester of formula (IV) via Mitsunobu reaction in the presence of acrylic acid or an acrylic acid analogue 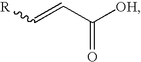
wherein R is H, (C₁-C₆)alkyl, or phenyl, in the presence of base; 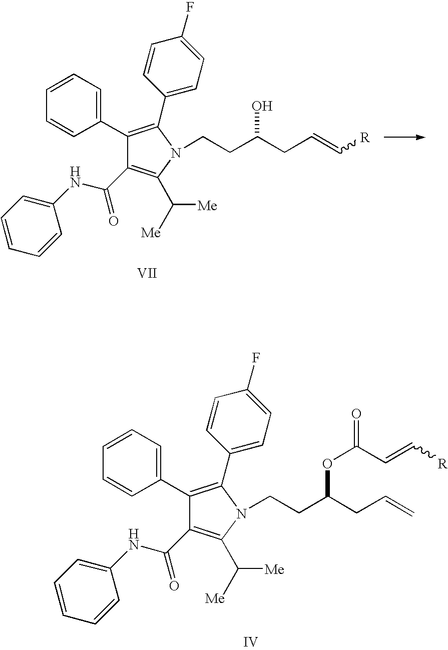
(c) contacting in a solvent the acryloyl ester (IV) with a catalyst to afford 5,6 dihydro pyran-2-one V; 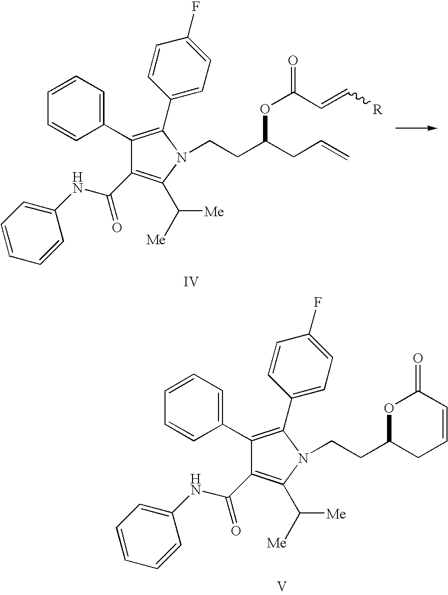
(d) converting the compound of formula (V) to a compound of formula (VI) via facial selective 1,4 addition of allyl or benzyl alcohol; 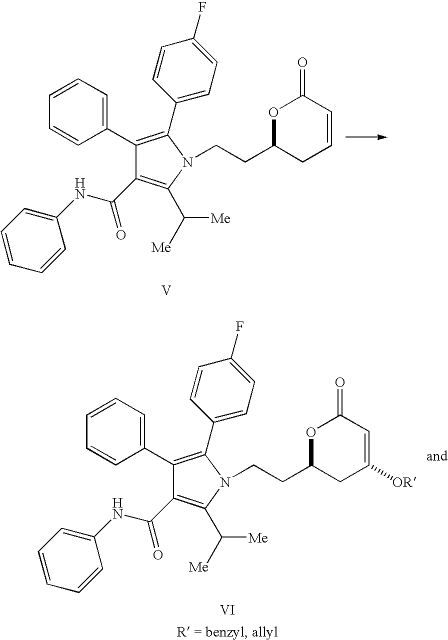
(e) removal of the allyl or benzyl moiety in the compound of formula (VI) via hydrogenolysis to give a compound of formula I.

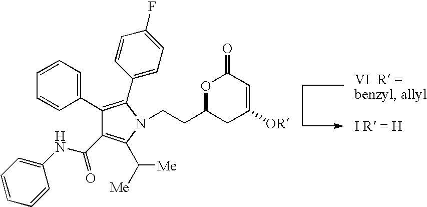

7. A process for the preparation of a compound of formula (I) 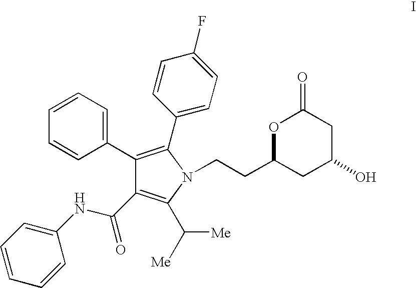
comprising:
(a) contacting in a solvent optionally in the presence of a Lewis acid a compound of formula (II) with 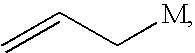
wherein M is SiCl₃, SiMe₃, B(OH)₂, CuLi, MgBr, ZnBr, InBr, SnR₃ wherein R₃ is (C₁-C₆)alkyl, to give a compound of formula (VIII): 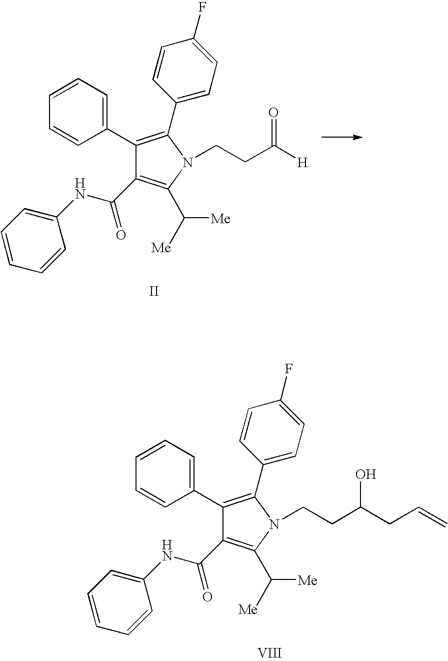
(b) isolating the desired enatiomer (VIII) from the enantiomeric mixture; 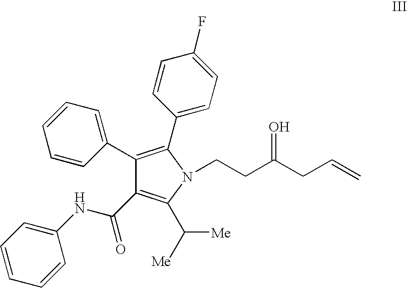
(c) conversion of the compound of formula (III) to an acryloyl ester of formula (IV) in the presence of base using 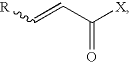
wherein X is Cl, Br, I, or 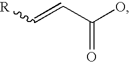
and R is H, (C₁-C₆)alkyl, or phenyl, or an acryloyl activated ester equivalent; 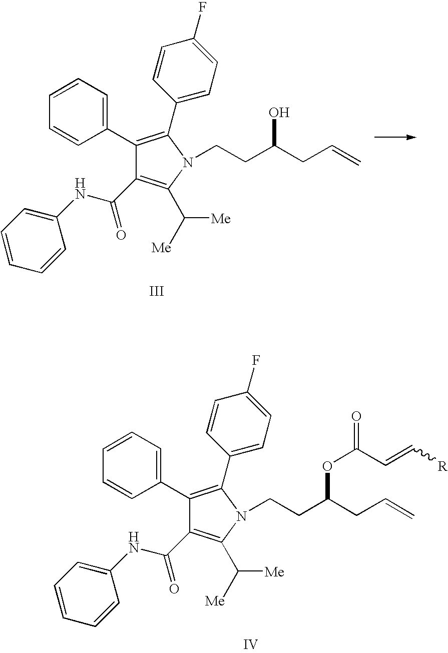
(d) contacting in a solvent the acryloyl ester (IV) with a catalyst to afford 5,6 dihydro pyran-2-one V; 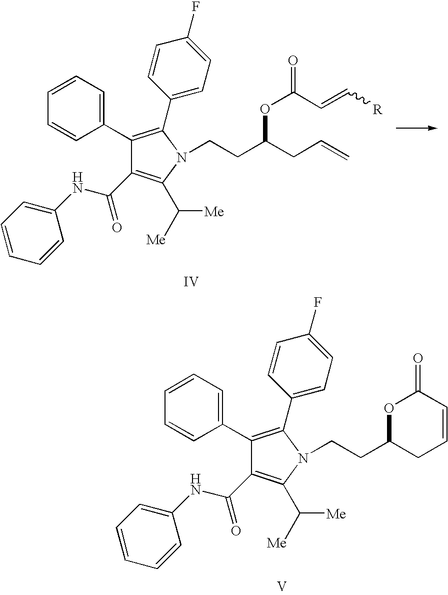
(e) converting the compound of formula (V) to a compound of formula (VI) via facial selective 1,4 addition of allyl or benzyl alcohol; 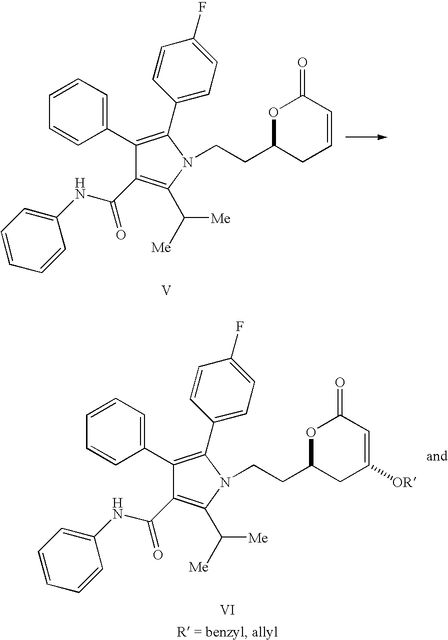
(f) removal of the allyl or benzyl moiety in the compound of formula (VI) via hydrogenolysis to give a compound of formula I.

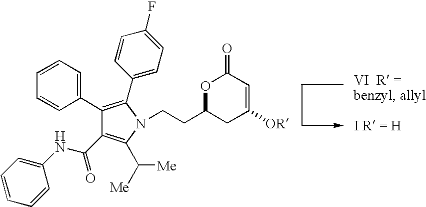

8. A process for the preparation of a compound of formula III 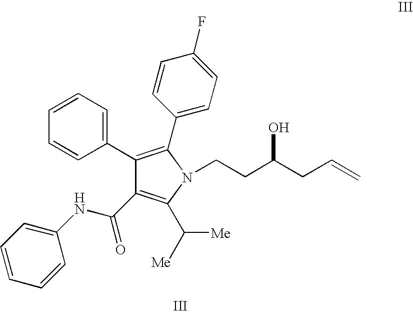
comprising:
(a) contacting a compound of formula (II) with an allenylboronic ester to give a compound of formula (XI): 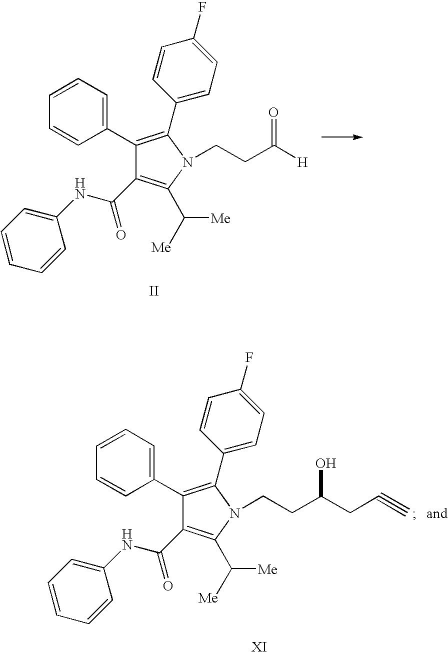
(b) hydrogenation of the compound of formula (XI) to provide III

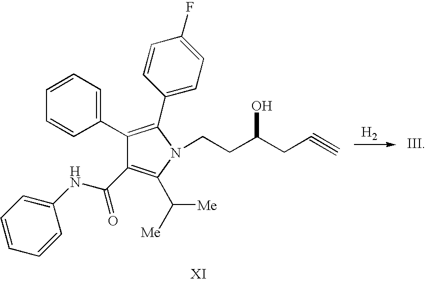

9. A process for the preparation of a compound of formula VII 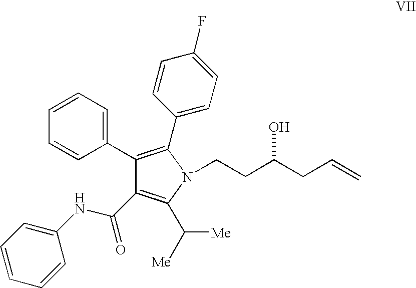
comprising:
(a) contacting contacting (II) with an allenylboronic ester to give a compound of formula (XII): 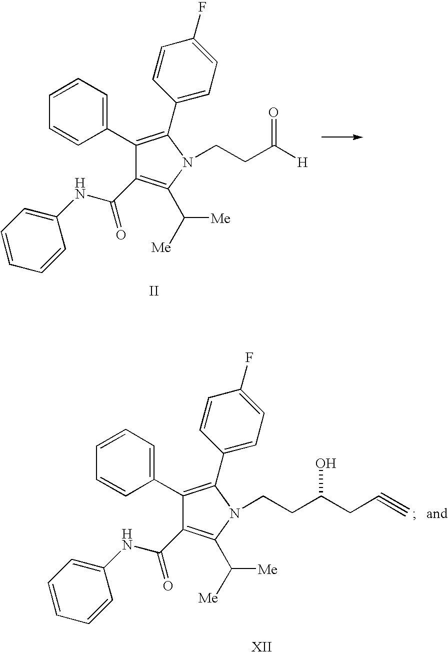
(b) hydrogenation of the compound of formula (XII) to provide VII

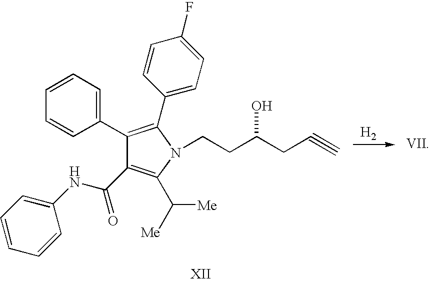

10. A process for the preparation of a compound of formula VIII 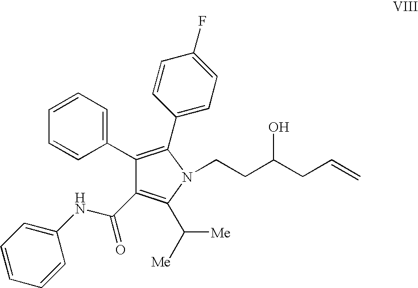
comprising:
(a) contacting (II) with allenylboronic acid or an allenylboronic ester to give a compound of formula (XIII): 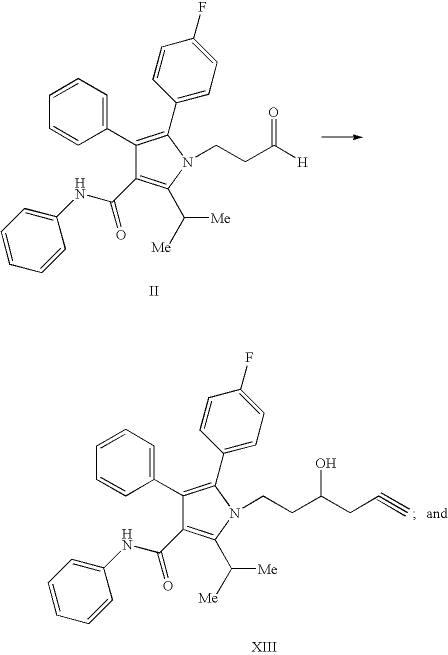
(b) hydrogenation of the compound of formula (XII) to provide VII

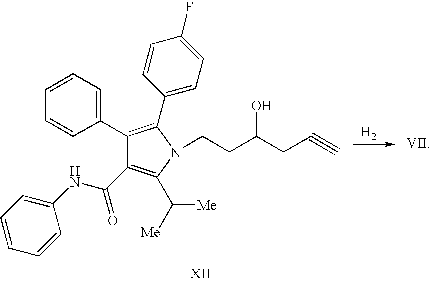

11. Compounds of the following formulas: 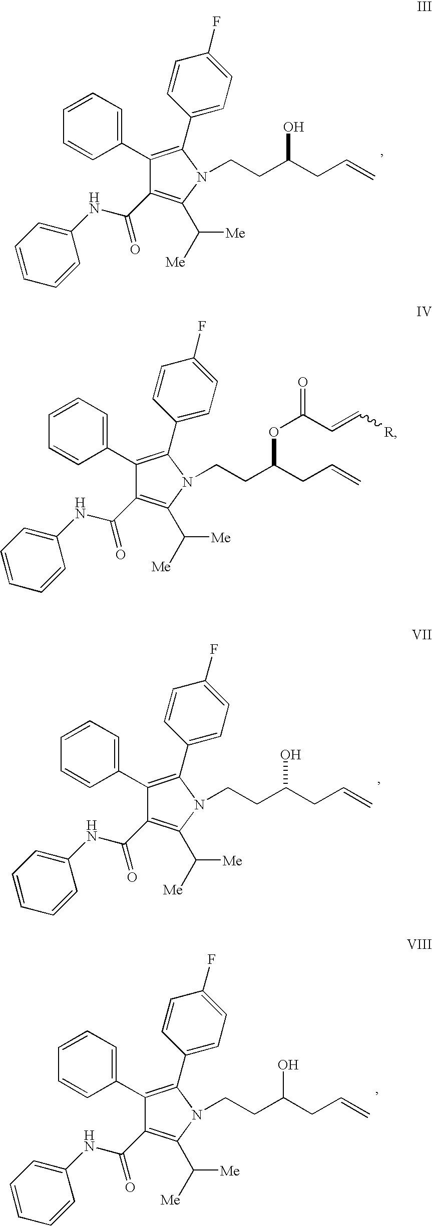 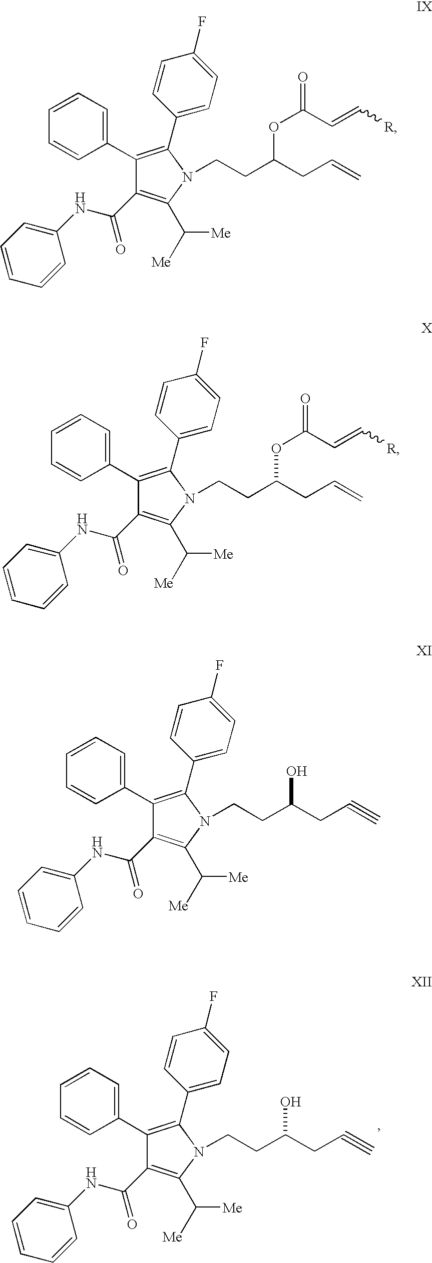 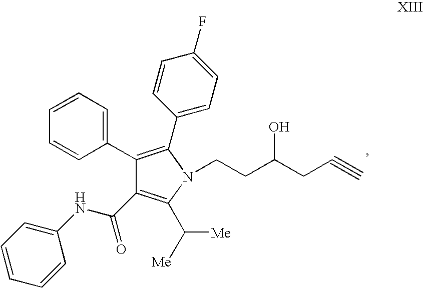
wherein R is H, (C₁-C₆)alkyl, or phenyl.
